(19) United States
(12) Patent Application Publication
(10) Pub. No.: US 2022/0192581 A1
(43) Pub. Date: Jun. 23, 2022

(54) SYSTEM AND METHOD FOR MONITORING AND RESPONDING TO A CEREBROVASCULAR ACCIDENT

(71) Applicants: KANEKA CORPORATION, Osaka-shi (JP); Kaneka Americas Holding, Inc., Pasadena, TX (US)

(72) Inventor: Hiromi Maeda, Newark, CA (US)

(73) Assignees: KANEKA CORPORATION, Osaka (JP); Kaneka Americas Holding, Inc., Pasadena, TX (US)

(21) Appl. No.: 17/599,337

(22) PCT Filed: Aug. 12, 2020
(86) PCT No.: PCT/US20/45895

§ 371 Date: Sep. 28, 2021

Related U.S. Application Data
(60) Provisional application No. 62/887,178, filed on Aug. 15, 2019.

(51) Int. Cl. A61B 5/00; G16H 50/30; G16H 10/60; G16H 10/20

(52) U.S. Cl. CPC A61B 5/4064; A61B 5/746; A61B 2562/0219; G16H 10/60; G16H 10/20; G16H 50/30

(57) ABSTRACT

A system for monitoring and responding to a cerebrovascular accident includes a computer, including a computer processor and a computer transceiver, a storage that stores personal medical information and a risk level, and a portable device attached to a user. When the computer transceiver receives personal medical information of the user, the computer processor calculates the risk level of a cerebrovascular accident based on the personal medical information. When the computer transceiver receives acceleration information, the computer processor determines whether to contact a predetermined contact based on the risk level and the personal medical information. The portable device includes an acceleration sensor, a position receiver, a clock, a portable transceiver, and a portable processor.

### TECHNICAL FIELD

One or more embodiments of the present invention relate to a system for monitoring and responding to cerebrovascular accident.

### BACKGROUND

Cerebrovascular accident, also known as “stroke,” generally includes both cerebral infarction and cerebral hemorrhage. The rate of occurrence of cerebral infarction and cerebral hemorrhage is about 7:3. The number of cerebrovascular accidents is about 800,000 per year in the United States alone. In particular, the rate of occurrence sharply increases after the age of 50. The number of people who have had a cerebrovascular accident in the past and are still alive reached a total of 7 million. Cerebrovascular accident is the fourth most common disease as a cause of death after heart disease, cancer, and pneumonia.

There are at least two points worth noting about a cerebrovascular accident. First, the recurrence rate is high. One fourth of cerebrovascular accident occurrence is due to recurrence. Second, two-thirds of people who have experienced a cerebrovascular accident have remained paralyzed, while more than half are suffering from severe paralysis.

Cerebral infarction occurs when a thrombus is generated and clogs the cerebral artery blood vessels. Therefore, an administration of a thrombolytic agent to dissolve the thrombus is performed as treatment. Alternatively, a physical capture and recovery of the thrombus using a metal mesh via special catheter guided to the infarct of the cerebral artery is performed when a large thrombus clogs a large-diameter blood vessel.

In the case of cerebral hemorrhage, surgical removal of leaked blood from the hemorrhage area via the skull (craniotomy) is performed as treatment. Alternatively, indwelling an implant called an embolization coil medically to the cerebral aneurysm via a catheter is performed when a mild condition such as blood exuding from an aneurysm of the cerebral artery blood vessel are performed as treatment.

Even though prompt surgical treatment is crucial in cerebral hemorrhage, the treatment cannot be performed if a physician determines that the treatment would damage any important tissues of the brain. Over time, the brain nerve tissue area that no longer receives blood (due to cerebral infarction) or that is compressed (due to intracranial pressure caused by cerebral hemorrhage) enlarges. Because loss of brain tissues is irreversible, paralysis may remain in people who do not receive prompt treatment. In an ambulance, people who are believed to have suffered a cerebrovascular accident receive tests on their way to a hospital to determine whether cerebrovascular accident occurred. After arriving at the hospital, a series of inspections such as CT and/or MRI examinations are performed on patients having a high possibility of experiencing a cerebrovascular accident.

Furthermore, treatments for cerebral infarction and cerebral hemorrhage are different. With cerebral infarction, because the thrombus must be dissolved, the first treatment performed is the administration of a thrombolytic agent. On the other hand, with cerebral hemorrhage, it is important to stop the leakage of blood from the cerebral blood vessels to the brain tissue, which is the opposite of improving blood flow, e.g., by administering a drug/agent. If by any chance the patient who suffers cerebral hemorrhage is misdiagnosed with cerebral infarction and administered thrombolytic agents as a result, the bleeding situation will worsen as the result of the blood becoming less viscous and difficult to coagulate when exposed to the thrombolytic agent. Therefore, for the treatment of cerebrovascular accident after onset, careful diagnosis for cerebral infarction or cerebral hemorrhage is also extremely important in addition to receiving prompt treatment.

Additionally, for patients diagnosed with cerebral infarction that have thrombus clogging in the large diameter vessel, such thrombus is too large and refractory to be dissolved by thrombolytic agents, and a specially designed catheter comprising a metal mesh described above is used to remove the large and refractory thrombus, which is an advanced medical treatment that requires a high degree of expertise and experience. The number of hospitals that can perform such the advanced medical treatment is limited.

As described above, sequelae of a cerebrovascular accident can be suppressed by performing appropriate and timely treatment. However, certain people have higher risk of cerebrovascular accidents, and this risk changes constantly and can only be recognized when they receive proper medical inspection. Therefore, if a cerebrovascular accident occurs without recognizing and managing the potential risk for cerebrovascular accidents, appropriate and timely treatment may not be possible. On the other hand, if the risk of experiencing a cerebrovascular accident is promptly recognized and managed, appropriate and timely treatment, or even prevention of the cerebrovascular accident, can be more easily achieved.

### SUMMARY

One or more embodiments of the present invention provide a system, a computer, and a portable device for monitoring and responding to cerebrovascular accident, and a method for monitoring and responding to cerebrovascular accident.

One or more embodiments provide a system for monitoring and responding to a cerebrovascular accident including a computer processor and a computer transceiver, wherein when the computer transceiver receives personal medical information of a user, the computer processor calculates a risk level of a cerebrovascular accident based on the personal medical information, and when the computer transceiver receives at least acceleration information, the computer processor determines whether to contact a predetermined contact based on the risk level and the user's personal medical information; a storage that stores the personal medical information and the risk level; a portable device attached to the user, including: an acceleration sensor that outputs acceleration user's abnormal movement signals; a position receiver that calculates position information of the user; a clock that outputs time information; a portable transceiver that transmits the position information, the acceleration information based on the acceleration user's abnormal movement signals, and the time information to the computer; and a portable processor that receives signals from the acceleration sensor, the position receiver, and the clock and that controls the portable transceiver. The computer, the storage, and the portable device are connected to each other via a computer network.

One or more embodiments provide a computer for monitoring and responding to a cerebrovascular accident, including: a computer processor that: receives personal medical information; and calculates a risk level of a cerebrovascular accident based on the personal medical information; and a computer transceiver, when receiving acceleration information from a portable device, and the computer processor determines whether to contact a predetermined contact based on the risk level and the acceleration information.

One or more embodiments provide a portable device attached to a user, including: an acceleration sensor that outputs acceleration signals; a position receiver that calculates position information of the user; a clock that outputs time information; a portable processor that obtains acceleration information from the acceleration sensor; a portable transceiver that transmits the position information, acceleration information based on the acceleration signals, and time to a computer; and an output device that outputs health information based on an instruction from the computer.

One or more embodiments provide a method for monitoring a cerebrovascular accident, including: receiving personal medical information by a computer; calculating a risk level of cerebrovascular accident based on the personal medical information by the computer; obtaining acceleration information based on acceleration signals from an acceleration sensor by a portable device; determining by the computer, based on the acceleration information and on the risk level, whether to contact predetermined contact; and contacting the predetermined contact when determining to contact predetermined contact.

### BRIEF DESCRIPTION OF THE DRAWINGS

FIG. 1 shows a system for monitoring and responding to a cerebrovascular accident connected with a peripheral system according to one or more embodiments.

FIG. 2 shows a hardware diagram of a computer according to one or more embodiments.

FIG. 3 shows a hardware diagram of a portable device according to one or more embodiments.

FIG. 4 shows a portable device according to one or more embodiments.

FIG. 5 shows a sequence and data-flow diagram of the system for personal medical information management, monitoring a cerebrovascular accident, accessing hospital database, and communication system with hospitals, fire stations, and relevant contacts according to one or more embodiments.

FIG. 6 shows an example of a risk factor table and personal medical information of cerebral infarction according to one or more embodiments.

FIG. 7 shows an example of a risk factor table and personal medical information of cerebral hemorrhage according to one or more embodiments.

FIG. 8 shows a flowchart of a sensor information process of the computer according to one or more embodiments.

FIG. 9 shows a flowchart of an emergency process of the computer according to one or more embodiments.

FIG. 10 shows a flowchart of a hospital selection process according to one or more embodiments.

FIG. 11 shows an example of the selected hospital information.

FIG. 12 shows a flowchart of an information update process of the computer according to one or more embodiments.

FIG. 13 shows examples of the personal medical information updated by the information from the portable device.

FIG. 14 shows examples of outputs of the portable device when the biological information or the acceleration information is received by the computer.

FIG. 15 shows messages on the portable device that are determined and displayed by a combination of risk factors.

### DETAILED DESCRIPTION

Specific embodiments of the invention will now be described in detail with reference to the accompanying figures. Like elements in the various figures are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

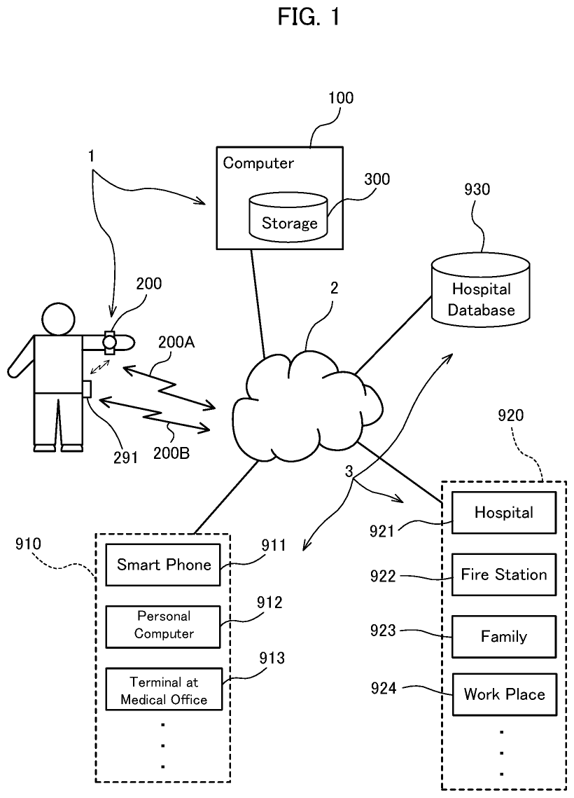

FIG. 1 shows a system 1 for monitoring and responding to a cerebrovascular accident and its peripheral system 3 according to one or more embodiments. The system 1 includes a computer 100, a portable device 200, and a storage 300. In one or more embodiments, the storage 300 is included in the computer 100, as shown in FIG. 1. The peripheral system 3 may include information terminals 910, terminals for predetermined contact 920, and a hospital database storage 930. Each of the computer 100, the portable device 200, the storage 300, the information terminals 910, the hospital database storage 930, and the terminals for predetermined contact 920 are connected through a computer network 2 (e.g. the Internet, etc.). In one or more embodiments, the storage 300 can be independent from the computer 100 (i.e., disposed externally of the computer 100) and may individually connect to the computer network 2.

In one or more embodiments, the portable device 200 can wirelessly connect to the computer network 2 as shown by signals 200A. Additionally or alternatively, the portable device 200 can wirelessly connect to the computer network 2 through a mobile device 291. The mobile device 291 may be a device, such as a smart phone, that can connect to the computer network 2 through a mobile communication network, and that can also wirelessly connect with the portable device 200 through WiFi® or Bluetooth®. The portable device 200 may be composed of a plurality of devices connected through wired or wireless communication with one another. In this case, the plurality of devices may include at least one of a wearable device, a smart phone, a display, and a biological sensor.

The portable device 200 is attached to a user. The storage 300 stores personal medical information and a risk level of the user. The information terminals 910 transmit the personal medical information to the computer 100 and can be at least one of a smart phone, a personal computer, and a terminal at a medical office. The predetermined contact 920 can include at least one of a hospital 921, a fire station 922 for ambulance, a family contact 923 of the user, and a work place contact 924 of the user.

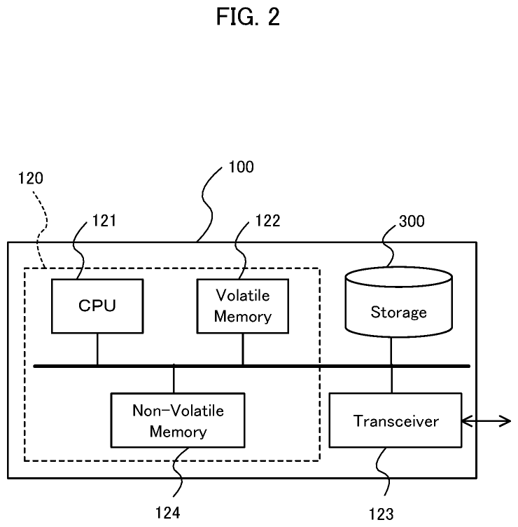

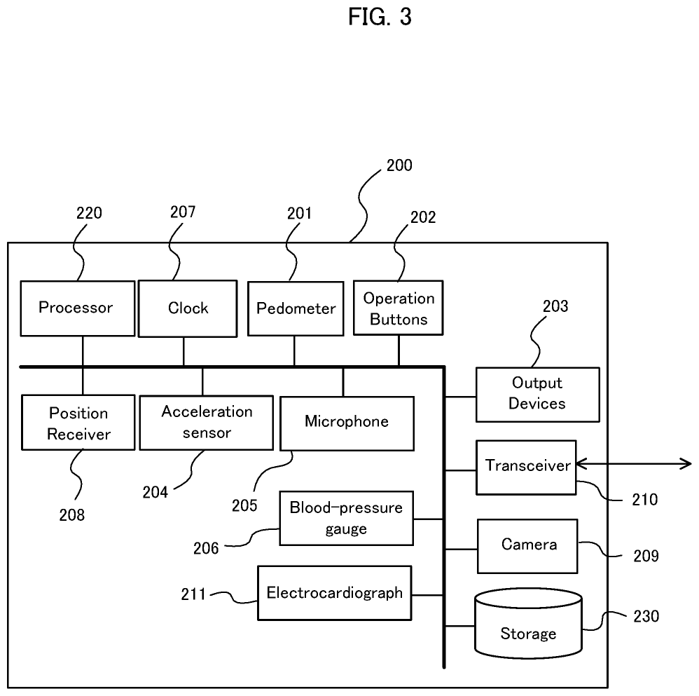

FIG. 2 shows a hardware diagram of the computer 100 with the storage 300 included in the computer 100 according to one or more embodiments. The computer 100 may be an information processing device that includes a storage 300, a CPU (central processing unit) 121, a volatile memory 122, a non-volatile memory 124, and a computer transceiver 123. The storage 300 may be non-volatile memory such as hard disks or flash memories etc., and stores the personal medical information and the risk level of a user. The volatile memory 122 may be RAM (random access memory) or cache memory, etc. The non-volatile memory 124 may be ROM (read only memory), flash memory, or hard disk, etc. The computer transceiver 123 connects to the computer network 2 to communicate with other devices that are also connected to the computer network 2. In the following description, the CPU 121, the volatile memory 122, the non-volatile memory 124, and the computer transceiver 123 are collectively referenced as a computer processor 120. However, it is also possible that the computer processor 120 includes only the CPU 121, or includes CPU 121 and any one or more of the volatile memory 122, the non-volatile memory 124, the computer transceiver 123, etc. The hardware configuration of the computer processor 120 shown in FIG. 2 is also applicable to the portable processor 220 of the portable device 200 as shown in FIG. 3.

The computer transceiver 123 receives personal medical information of the user, and the computer processor 120 calculates a risk level of a cerebrovascular accident based on the personal medical information. When the computer transceiver 123 receives acceleration information (described below), the computer processor 120 determines whether to contact predetermined contact 920 based on the calculated risk level and the user's personal medical information, and the computer transceiver 123 contacts the predetermined contact 920 based on the determination.

FIG. 3 shows a hardware diagram of the portable device 200 according to one or more embodiments. The portable device 200 includes a portable processor 220, a portable transceiver 210, a position receiver 208, a clock 207, a portable storage 230, an acceleration sensor 204, output devices (display/speaker) 203, and a plurality of operation buttons 202. The portable device 200 may further include a biological sensor that transmits biological information of the user such as: a pedometer 201, a microphone 205, a blood-pressure gauge 206, an electrocardiograph 211, a camera 209, etc.

The portable processor 220 may include not only CPU but also volatile and non-volatile memory as described above. The portable processor 220 receives signals from the acceleration sensor 204, the position receiver 208, and the clock 207, and controls the portable transceiver 210. The portable transceiver 210 transmits and receives data from the computer network 2. Additionally or alternatively, the portable transceiver 210 may connect to the computer network 2 through the mobile device 291. The acceleration sensor 204 outputs user's abnormal movement signals. The position receiver 208 calculates position information of the user based on data received from, for example, a Global Navigation Satellite System (GNSS). The clock 207 outputs time information. The portable transceiver 210 transmits the biological information, the position information, the acceleration information based on the user's abnormal movement signals, and the time to the computer 100 through the computer network 2.

The portable storage 230 may store the personal medical information to be transmitted when the portable transceiver 210 cannot connect directly or indirectly to the computer network 2 and/or store information copied from the storage 300 to be utilized in the portable device 200. The output devices 203 may include one or more of a display, a speaker, and/or a vibration device to notify the user of information from the computer 100. The operation buttons 202 can accept a user's operation such as an emergency operation and a cancel operation, which are described below. The pedometer 201 accumulates the number of steps of the user. The microphone 205 senses the user's voice and converts it to digital voice signals. The blood-pressure gauge 206 measures the blood pressure of the user. The electrocardiograph 211 measures the user's heart beat rate. The camera 209 can take digital pictures of the user's face.

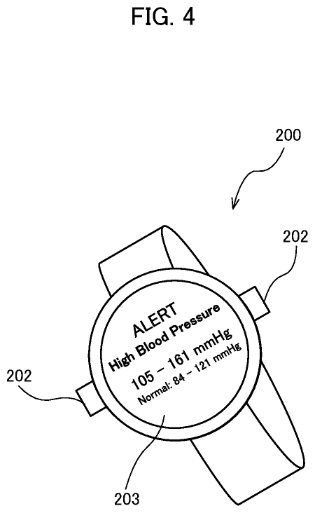

FIG. 4 shows a portable device 200 according to one or more embodiments. As shown in FIG. 4, the portable device 200 may be in the shape of a wristwatch. Alternatively, the portable device 200 may be in the form of any wearable items such as a necklace, a piercing, a skin attachment, an earplug, an eyeglass, a belt attachment, a shirt attachment, a trouser attachment, etc. In FIG. 4, only the output device 203 (e.g., a display) and the operation buttons 202 are shown on an external surface of the portable device 200.

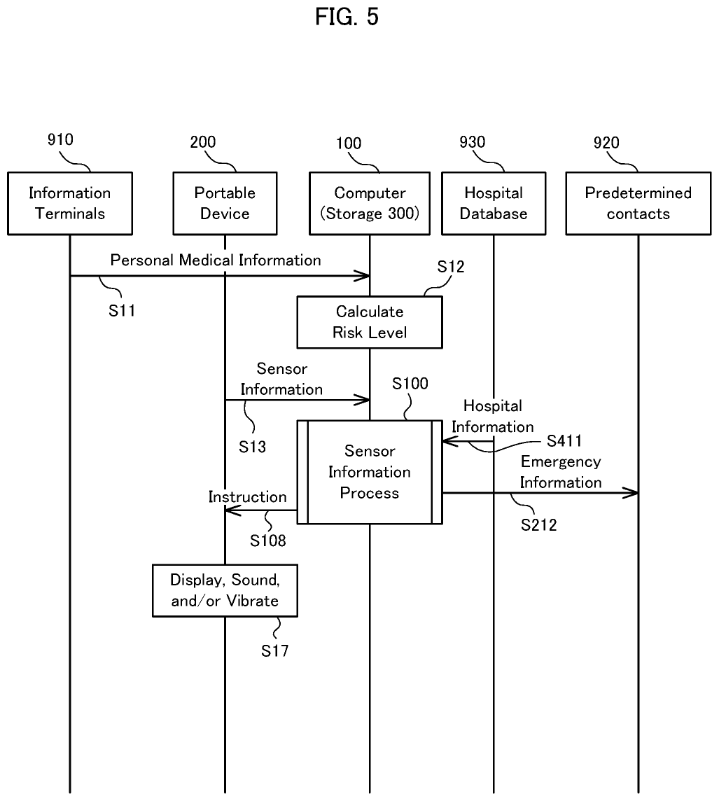

FIG. 5 shows a sequence and data-flow diagram of the system 1 for managing personal medical information, monitoring a cerebrovascular accident, accessing hospital database, and communicating with hospitals, fire station, and relevant contacts according to one or more embodiments. As shown FIG. 5, one of the information terminals 910 transmits the personal medical information of a user to the computer 100 (Step S11). The personal medical information may include at least one of medical record information received from a medical office, a health check result received from a medical office, and a genetic test result received from a genetic testing company.

In one or more embodiments, the computer processor 120 causes the output device 203 to output questions for the user. When the user answers the questions, the portable transceiver 210 transmits the answers to the computer processor 120. When the computer processor 120 receives the answers, the computer processor 120 stores the answers in the storage 300 as a part of the personal medical information.

When the computer 100 receives the personal medical information, the computer processor 120 calculates the risk level of cerebrovascular accident using the personal medical information (Step S12). If any personal medical information has been previously stored in the storage 300 before the computer processor 120 receives new personal medical information, the computer processor 120 may add the received personal medical information to the stored personal medical information and calculate or recalculate the risk level of cerebrovascular accident based on the integrated personal medical information that includes both the previously-stored and newly-received personal medical information. Alternatively, the computer processor 120 may prioritize calculating the risk level using only the most recent (i.e., newest) personal medical information.

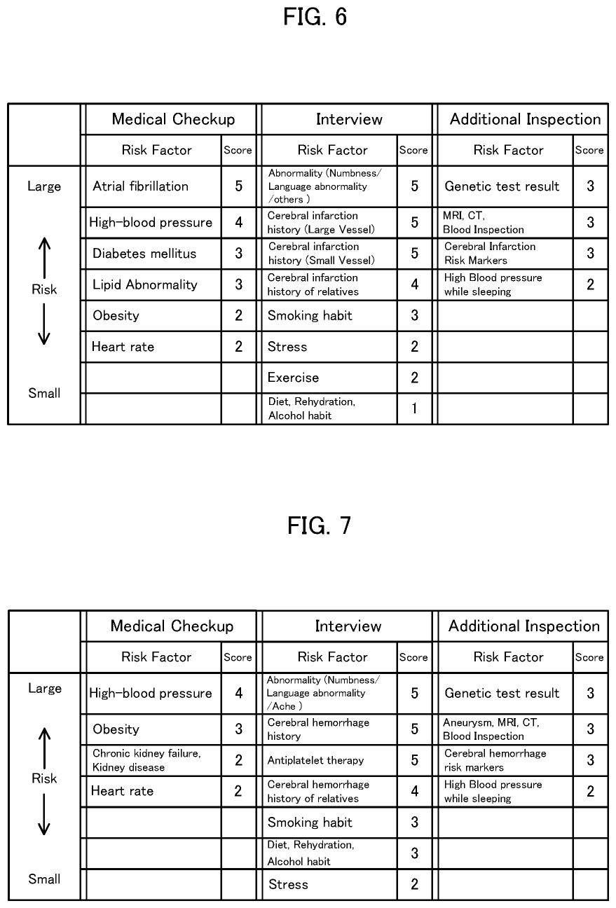

FIG. 6 shows an example of a risk factor table and the personal medical information of cerebral infarction according to one or more embodiments. As shown in FIG. 6, risk factors relating to cerebral infarction are listed together with a score assigned to each risk factor. The score could be larger number as a risk (i.e., chance of occurrence) of cerebral infarction increases. The risk factors relating to cerebral infarction could include, for example, atrial fibrillation, high-blood pressure, diabetes mellitus, lipid abnormality, obesity, heart rate, abnormalities such as numbness and language abnormality, cerebral infarction history (large vessel), cerebral infarction history (small vessel), cerebral infarction history of relatives, smoking habit, stress, exercise, diet rehydration, alcohol habit, genetic test result, MRI result, CT result, blood inspection, cerebral infarction risk markers, and high blood pressure while sleeping, etc. Each of the risk factors may be determined as “applicable” by the computer processor 120 when symptoms corresponding to the risk factor are observed from the user or when measurement value corresponding to the risk factor is within a predetermined range.

FIG. 7 shows an example of a risk factor table and personal medical information of cerebral hemorrhage according to one or more embodiments. As shown in FIG. 7, risk factors relating to cerebral hemorrhage are listed together with a score assigned to each risk factor. The score could be larger number as a risk (i.e., chance of occurrence) of cerebral hemorrhage increases. The risk factors relating to cerebral hemorrhage could include, for example, high-blood pressure, chronic kidney failure, kidney disease, obesity, heart rate, abnormalities such as numbness and language abnormality, cerebral hemorrhage history, antiplatelet therapy, cerebral hemorrhage history of relatives, smoking habit, stress, genetic test result, MRI result, CT result, blood inspection, aneurysm, cerebral hemorrhage risk markers, and high blood pressure while sleeping, etc. Each of the risk factors may be determined as “applicable” by the computer processor 120 when symptoms corresponding to the risk factor are observed from the user or when the measurement value corresponding to the risk factor is within a predetermined range.

The information that makes up each personal medical information can be obtained from various sources such as the medical record information or the portable device 200. Each personal medical information may be stored together with a corresponding date and time stamp as archival record. Alternatively, each personal medical information may be stored together with its corresponding source of information and/or the corresponding location from which the personal medical information is obtained.

The user's risk level of cerebral infraction and cerebral hemorrhage may be calculated independently or in combination. The risk level may be calculated by various methods, including weighted average. In this example, the risk level is based on the simple average of the scores of each risk factors whose symptom are observed on the user or whose measurement values of the user are within a predetermined range. In one or more embodiments, the risk level may be raised when a predetermined combination of risk factors is simultaneously applicable to a user. The computer processor 120 can also determine whether there is a higher risk of cerebral infarction or cerebral hemorrhage using the calculated risk levels.

Returning to FIG. 5, when the computer transceiver 123 receives sensor information from the acceleration sensor 204 or one of the biological sensors of the portable device 200 (Step S13), the computer processor 121 performs the sensor information process S100. Here, the sensor information may include information relating to an emergency operation, which is described below in more detail. When the portable device 200 receives an instruction from the computer 100 (Step S108), the output device 203 outputs health information based on the instruction from the computer 100 (Step S17).

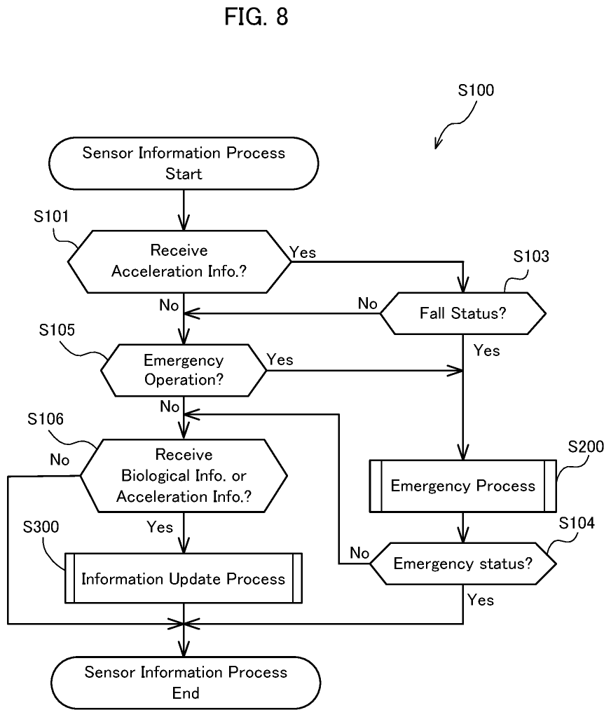

FIG. 8 shows a flowchart of the sensor information process S100 implemented by the computer 100 according to one or more embodiments. The computer processor 120 determines whether the received sensor information is the acceleration information (Step S101). When the computer processor 120 determines that the received sensor information is the acceleration information (Step S101: Yes), the computer processor 120 further determines whether the acceleration information indicates a fall status that indicates that the user has fallen down (Step S103). When the computer processor 120 determines that the acceleration information indicates a fall status (Step S103: Yes), the computer processor 120 proceeds to implement the emergency process S200.

When the computer processor 120 determines that the received sensor information is not the acceleration information (Step S101: No) or that the acceleration information does not indicate a fall status (Step S103: No), the computer processor 120 further determines whether the emergency operation on the portable device 200 has been triggered by the user (Step S105). When the computer processor 120 determines that the emergency operation was triggered (Step S105: Yes), the computer processor 120 proceeds to implement the emergency process S200. When the computer processor 120 determines that the emergency operation was not triggered (Step S105: No), the computer processor 120 proceeds to step S106. In one or more embodiments, one example of the emergency operation may be to hold down the predetermined plurality of operation buttons 202 of the portable device 200. When the predetermined plurality of operation buttons 202 are held down, the portable transceiver transmits the emergency operation information to the computer 100.

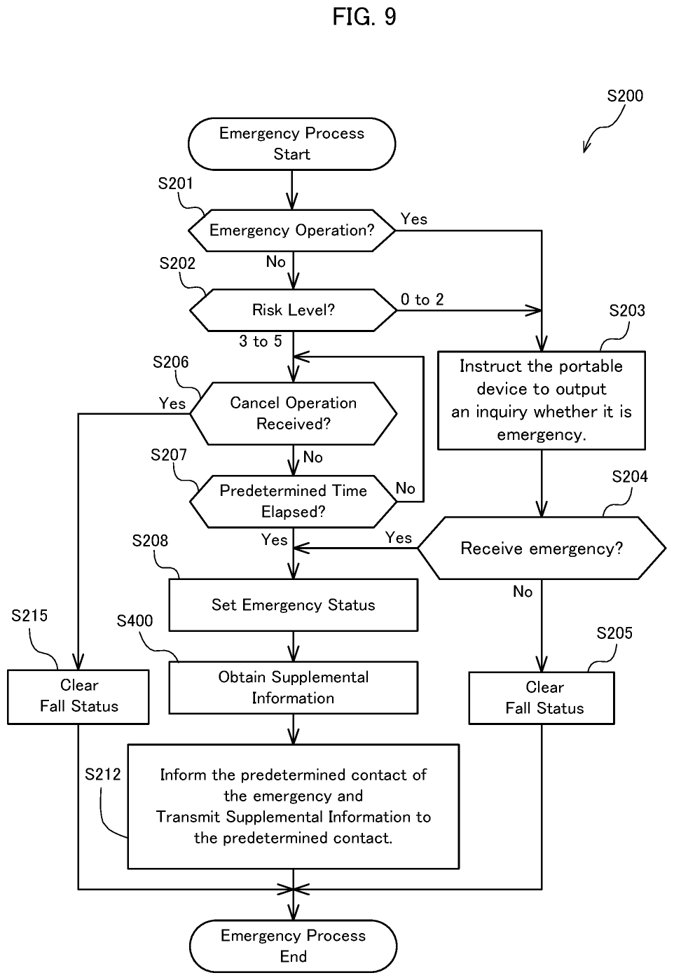

After the completion of the emergency process S200, which is described in more detail below in reference to FIG. 9, the computer processor 120 confirms whether there is an emergency status (Step S104). If there is an emergency status (Step S104: Yes), the computer processor 120 completes the sensor information process S100. In the case that it is not an emergency status (Step S104: No), the computer processor 120 proceeds to step S106.

In step S106, the computer processor 120 confirms whether the computer 100 receives the biological information or the acceleration information (Step S106). When the computer processor 120 cannot confirm whether the biological information or the acceleration information was received (Step S106: No), the computer processor 120 finishes the sensor information process S100. When the computer processor 120 confirms that the computer 100 received the biological information or the acceleration information (Step S106: Yes), the computer processor 120 proceeds to the information update process S300. After the completion of the information update process S300, the computer processor 120 finishes the sensor information process S100.

FIG. 9 shows a flowchart of the emergency process S200 implemented by the computer 100 according to one or more embodiments. As shown in FIG. 9, the computer processor 120 confirms whether an emergency operation was triggered by the user on the portable device 200 (Step S201). When the computer processor 120 confirms that the emergency operation was triggered (Step S201: Yes), the computer processor 120 proceeds to step S203. When the computer processor 120 confirms that the emergency operation was not triggered (Step S201: No), the computer processor 120 further confirms the risk level of cerebrovascular accident for the user (Step S202). When the risk level of cerebrovascular accident is between 3 to 5 (Step S202: 3 to 5), the computer processor 120 further determines whether a cancel operation has been performed by the user (Step S206). In one or more embodiments, when the risk levels are separately calculated for cerebral infarction and cerebral hemorrhage, the computer processor 120 may proceed to step S206 when either risk level is between 3 to 5.

When the computer processor 120 confirms that the cancel operation is performed by the user (Step S206: Yes), the fall status is cleared and the emergency process S200 is completed. When the computer processor 120 confirms that the cancel operation is not performed by the user (Step S206: No), the computer processor 120 confirms whether a predetermined time has elapsed (Step S207). When the computer processor 120 confirms that the predetermined time has not elapsed (Step S207: No), the computer processor 120 repeats step S206. When the computer processor 120 confirms that the predetermined time has elapsed (Step S207: Yes), the computer processor 120 proceeds to step S208. As described above, when the computer transceiver 123 receives the cancel operation information, the computer processor 120 can stop a process to contact the most suitable hospital(s) and/or the predetermined contact 920. Here, when the portable processor 220 receives a cancel operation from the user, the portable transceiver 210 transmits the cancel operation information.

When the risk level of cerebrovascular accident is between 0 to 2 (Step S202: 0 to 2), the computer transceiver 123 transmits an instruction to cause the output device 203 to output an inquiry to the user to confirm whether an emergency has occurred (Step S203). In one or more embodiments, when the risk levels are separately calculated for cerebral infarction and cerebral hemorrhage, the computer processor 120 may proceed to step S203 when both risk levels are between 0 to 2. When the computer transceiver 123 receives a response from the user that an emergency has not occurred (Step S204: No), the fall status is cleared and the emergency process S200 is completed. When the computer transceiver 123 receives a response from the user that emergency has occurred (Step S204: Yes), the computer processor 120 proceeds to step S208.

In step S208, the computer processor 120 sets the emergency status and obtains supplemental information about the user (Step S400). Then, the computer transceiver 123 informs at least one of the predetermined contacts 920 of the emergency status and transmits the supplemental information together with the position information and the time to the most suitable hospital(s) and/or the predetermined contact 920 (Step S212) when the computer processor 120 contacts the predetermined contact. After step S212, the emergency process S200 is completed.

Relating to step S400, the computer processor 120 may further determine which possibility is higher, of cerebral infarction or of cerebral hemorrhage, based on the personal medical information. In this case, the supplemental information may include the results of this determination to indicate which has a higher possibility of occurrence, cerebral infraction or cerebral hemorrhage.

The computer processor 120 may further determine whether an emergency status occurs at the user's residence by comparing the user's position information with the user's physical address. When the computer processor 120 determines that the emergency status occurs at the user's residence, the computer transceiver 123 may transmit at least one of the information about the user's residence and the accessibility of the user's residence as the supplemental information. In this case, the storage 300 may also store at least one of a physical address of the user, a commuting route to the user's residence, information about the user's residence, and accessibility of the user's residence. In the case that the supplemental information includes information relating to the accessibility of the user's residence, it would become easier for the ambulance crew to access the user's residence at the time of an emergency.

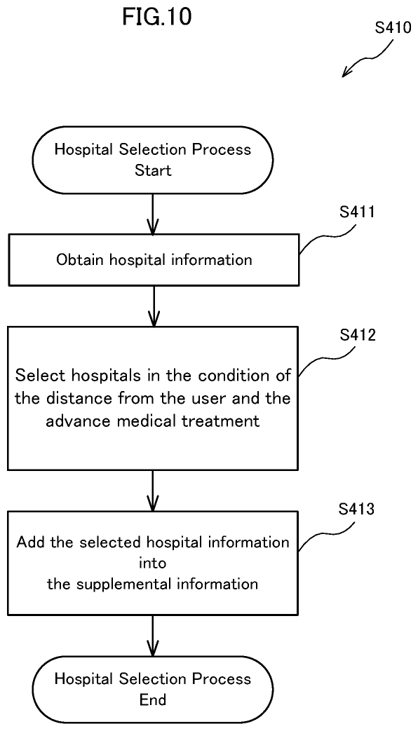

In one or more embodiments, the supplemental information may include information based on the hospital information stored in the hospital database storage 930. FIG. 10 shows an exemplary flowchart of the hospital selection process S410 according to one or more embodiments. The computer processor 120 obtains the hospital information from the hospital database storage 930 through the computer transceiver 123 (Step S411). Next, the computer processor 120 selects one or more appropriate hospitals based on the obtained hospital information (Step S412).

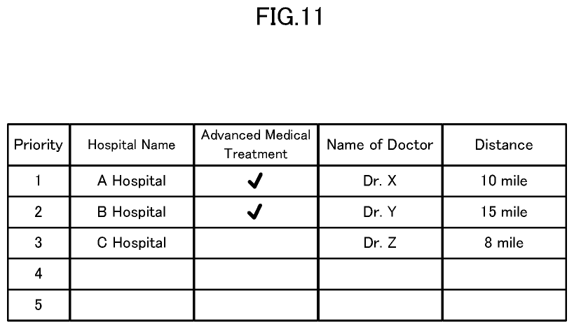

FIG. 11 shows an example of the selected hospital information. As shown in FIG. 11, the computer processor 120 extracts and lists all hospitals from hospital data that are ready to urgently receive patients with cerebral infarction or cerebral hemorrhage near the place where an emergency event occurred, selects hospitals, from among the hospital list, that are closest in distance to the user when the computer processor 120 determines that the personal medical information indicates that the user does not have any history of cerebral infarction or cerebral hemorrhage, or the user does not have any history of cerebral infarction in the large vessel even if the user had cerebral infarction in the past. In this case, the computer transceiver 123 may transmit hospital data of the closest hospitals as the supplemental information to the predetermined contact 920. The computer processor 120 may also select hospitals that are capable of providing the advanced medical treatments with higher priority than geographical proximity when the user's personal medical information indicates that the user has a history of having cerebral infarction at the large vessel or that the user may be suspected for cerebral infarction at the large vessel. When there is a high possibility that the user will require the advanced medical treatment, being able to select hospitals that are able to provide such advanced medical treatments can be extremely beneficial for the user. As shown in FIG. 11, the computer processor 120 may provide a priority order for the selected hospitals tailored to the user. In one or more embodiments, the nearest hospital that is capable of providing the advanced medical treatment is assigned the highest priority. To be able to make this hospital selection, the hospital database storage 930 may store hospital data that includes at least one of a name of the hospital, a location of the hospital, repeatedly updated hospital's availability for urgent treatment at that time, a possibility of the hospital having advanced medical treatment options, and a name of a doctor working at the hospital.

Returning to FIG. 10, the computer processor 120 adds the selected hospital information into the supplemental information (Step S413). The computer transceiver transmits the hospital data of the nearest hospitals as the supplemental information. The selected hospital information could be used by any individual (e.g., a fireman, an ambulance crew, an emergency responder, etc.) that takes the user to a hospital.

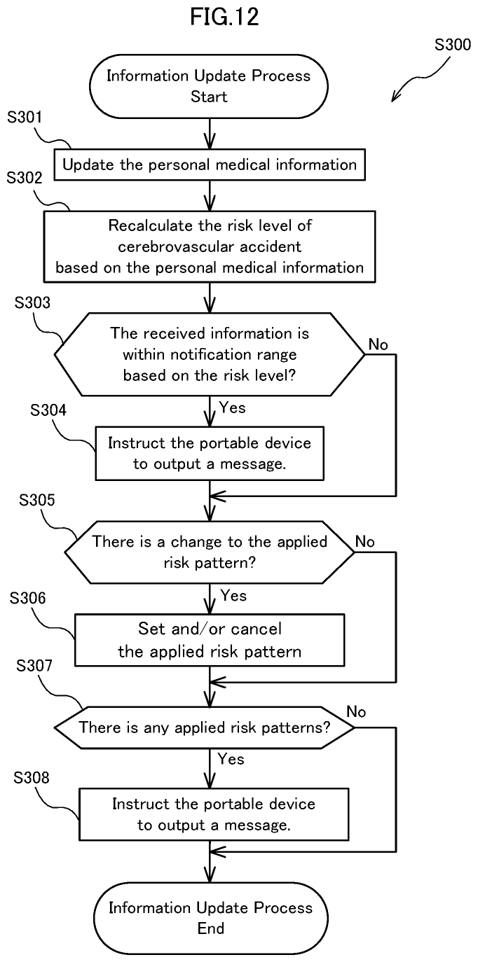

FIG. 12 shows a flowchart of the information update process S300 according to one or more embodiments. The computer processor 120 updates the personal medical information to include the biological information or the acceleration information when the computer processor 120 receives the biological information or the acceleration information of the user (Step S301). The computer processor 120 then recalculates the risk level based on the updated personal medical information (Step S302). The calculation method described in step S12 of FIG. 5 may also be employed in the recalculation method in step S302 of the information update process S300.

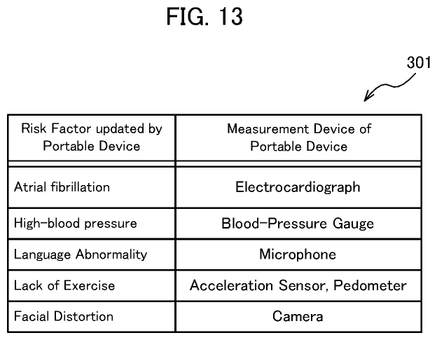

FIG. 13 shows examples of the personal medical information updated by the information from the portable device 200. The risk factor “atrial fibrillation” may be updated using information (e.g., signals) obtained from the electrocardiograph 211 of the portable device 200. The risk factor “high-blood pressure” may be updated using information (e.g., signals) obtained from the blood-pressure gauge 206 of the portable device 200. The risk factor “language abnormality” may be updated using information (e.g., signals) obtained from the microphone 205 of the portable device 200. The risk factor “exercise” may be updated based on information (e.g., signals) from the acceleration sensor 204 and/or the pedometer 201 of the portable device 200. The risk factor “facial distortion” may be updated based on information from the camera 209. The information (e.g., signals) from the biological sensor and the acceleration sensor 204 may be processed by the computer processor 120 before being stored in the storage 300.

Returning to step S303 of FIG. 12, the computer processor 120 confirms whether the received biological information and/or the received acceleration information are within the predetermined notification range based on the risk level (Step S303). When the computer processor 120 confirms that the received biological information and/or the received acceleration information are not within the predetermined notification range based on the risk level (Step S303: No), the computer processor 120 proceeds to step S305. When the computer processor 120 confirms that the received biological information and/or the received acceleration information is within the predetermined notification range based on the risk level (Step S303: Yes), the computer transceiver 123 transmits an instruction to cause the output device 203 to output a message to the user (Step S304). The output message may be predetermined depending on the received biological information and/or the received acceleration information along with the user's risk level. The computer processor 120 then proceeds to step S305. The predetermined notification range may be a predetermined applicable range of the risk factor such as a predetermined high-blood pressure range, a predetermined irregular heartbeat range, a predetermined range of a lack of exercise, etc. The predetermined notification range may be stored in the storage 300 or the non-volatile memory 124 of the computer 100.

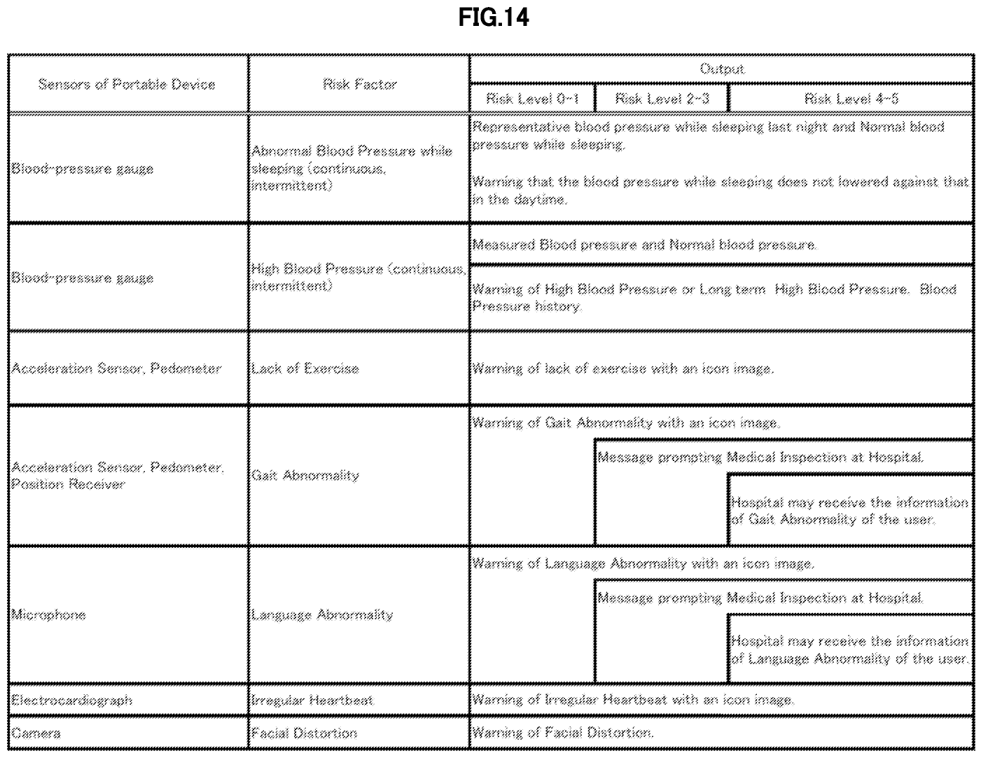

FIG. 14 shows examples of outputs that can be output to (e.g., displayed on) the output device 203 when the biological information and/or the acceleration information is received by the computer 100. The computer processor 120, using the signals from the blood-pressure gauge 206, determines that a user's blood pressure is abnormal (continuously or intermittently) while the user is sleeping when the user's blood pressure while sleeping is not lower compared to the user's average blood pressure during the day time. In response, the computer transceiver 123 transmits an instruction that causes the output device 203 to output (e.g., display) a warning that the user's blood pressure has not lowered while the user is sleeping. The output device 203 may further display the representative blood pressure while sleeping the previous night and the normal blood pressure while sleeping. In this example, although the output content is not different among each of the risk levels, the output content could be differentiated between different risk levels.

In another example, the computer processor 120 determines that a user's blood pressure is high (continuous or intermittent) when a blood pressure indicated by the blood-pressure gauge 206 is within a notification blood-pressure range according to the signals from the blood-pressure gauge 206. In response, the computer transceiver 123 transmits an instruction that causes the output device 203 to display a warning of high blood pressure (when the high blood pressure is intermittent) or long term high blood pressure (when the high blood pressure is continuous). The output device 203 may further display a measured blood pressure and a normal blood pressure as shown in FIG. 4. In this example, although the output content is not different among each of the risk levels, the output content could be differentiated between different risk levels.

In another example, when the computer processor 120 determines a lack of exercise when a value indicated by the pedometer 201 is within a notification range, the computer transceiver 123 transmits an instruction that causes the output device 203 to display a warning of lack of exercise with an icon image. The computer processor 120 may determine lack of exercise based on the output of the acceleration sensor 204 instead of the pedometer 201. In this example, although the output content is not different among each of the risk levels, the output content could be differentiated between different risk levels.

In another example, when the computer processor 120 determines a gait abnormality based on the output of the acceleration sensor 204, the pedometer 201, and/or the position receiver 208, the output device 203 may display a warning of the gait abnormality with an icon image. When the risk level of the user is between 2 to 5, the output device 203 may further display a message prompting the user to get a medical inspection at a hospital. When the risk level of the user is between 4 to 5, the computer transceiver 123 may further inform the user's hospital of the gait abnormality.

In another example, when the computer processor 120 determines a language abnormality based on the output of the microphone 205, the output device 203 may display a warning of the language abnormality with an icon image. When the risk level of the user is between 2 to 5, the output device 203 may further display a message prompting the user to take get a medical inspection at a hospital. When the risk level of the user is between 4 to 5, the computer transceiver 123 may further inform the user's hospital of the language abnormality.

In another example, when the computer processor 120 determines an irregular heartbeat based on the output of the electrocardiograph 211, the output device 203 may display a warning of the irregular heartbeat with an icon image. In this case, although the output content is not different among each of the risk levels, the output content could be differentiated between different risk levels.

In another example, when the computer processor 120 determines a facial distortion based on the output of the camera 209, the output device 203 may display a warning of the facial distortion with an icon. In this case, although the output content is not different among each of the risk levels, the output content could be differentiated between different risk levels.

The above-described determination of various symptoms by the computer processor 120 may be conducted independently and corresponding warnings may be displayed on the output device 203 in sequence.

Returning to step S305 of FIG. 12, the computer processor 120 determines whether there is a change in the applied risk pattern. Each risk pattern is associated with one or more risk factors. The risk pattern is introduced in a case that all or some of the risk factors associated with each risk pattern are applicable to a user. The detail of the risk patterns will be described below. When the computer processor 120 confirms that there is not a change to the applied risk pattern (Step S305: No), the processor proceeds to step S306. When the computer processor 120 confirms that there is a change to the applied risk pattern (Step S305: Yes), the computer processor 120 sets or cancels the applied risk pattern (Step S306) and proceeds to step S307.

In step S307, the computer processor 120 confirms whether there is an applied risk pattern. When the computer processor 120 confirms that there are no applied risk patterns (Step S307: No), the computer processor 120 finishes the information update process S300. When the computer processor 120 confirms that there is an applied risk pattern (Step S307: Yes), the computer transceiver 123 transmits an instruction that causes the output device 203 to output a notification message to the user (Step S308). The message may be predetermined based on the applied risk pattern and the user's risk level. Then, the computer processor 120 finishes the information update process S300. When a risk pattern is applied to the user, the computer processor 120 may periodically transmit an instruction based on the risk pattern.

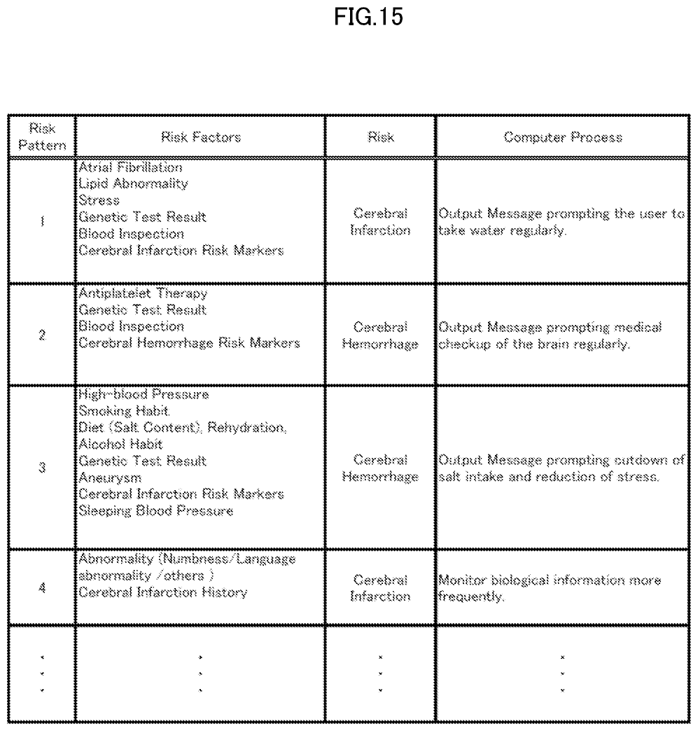

FIG. 15 shows risk patterns that are indicated by a combination of risk factors. Each risk pattern is associated with one or more risk factors. When all or some of the risk factors of each risk pattern are applicable to a user, the computer processor 120 determines that the risk pattern is introduced to the user. Risk pattern 1 indicates a risk of cerebral infarction. Risk pattern 1 is determined using the combination of risk factors including atrial fibrillation, lipid abnormality, stress, genetic test result, blood inspection, and cerebral infarction risk markers. When risk pattern 1 is applicable to the user, the computer transceiver 123 may regularly transmit an instruction to cause the output device 203 to output a message prompting the user to often intake water.

Risk pattern 2 indicates a risk of cerebral hemorrhage. Risk pattern 2 is determined using the combination of risk factors including antiplatelet therapy, genetic test result, blood inspection, and cerebral hemorrhage risk markers. When risk pattern 2 is applicable to the user, the computer transceiver 123 may regularly transmit an instruction to cause the output device 203 to output a message prompting the user to take a medical checkup of the user's brain.

Risk pattern 3 in FIG. 15 indicates a risk of cerebral hemorrhage. Risk pattern 3 is determined using the combination of risk factors including high-blood pressure, smoking habit, diet (salt content), rehydration, alcohol habit, genetic test result, aneurysm, cerebral infarction risk markers, and sleeping blood pressure. When risk pattern 3 is applicable to the user, the computer transceiver 123 may regularly transmit an instruction to cause the output device 203 to output a message prompting the user to cut salt intake or to reduce stress.

Risk pattern 4 in FIG. 15 indicates a risk of cerebral infarction. Risk pattern 4 is determined using the combination of risk factors of abnormality (numbness/language abnormality/others) and cerebral infarction history. When risk pattern 4 is applicable to the user, the computer processor 120 monitors the user's biological information more frequently.

In the above-described embodiments, although the computer 100 is described as a single device, the computer 100 may be a plurality of computers (computer devices) that are connected to the computer network 2.

Although the disclosure has been described with respect to only a limited number of embodiments, those skilled in the art, having benefit of this disclosure, will appreciate that various other embodiments may be devised without departing from the scope. Accordingly, the scope of the invention should be limited only by the attached claims.

1. A system for monitoring and responding to a cerebrovascular accident, comprising:
a computer comprising a computer processor and a computer transceiver,
wherein, when the computer transceiver receives personal medical information of a user, the computer processor calculates a risk level of a cerebrovascular accident based on the personal medical information, and
wherein, when the computer transceiver receives acceleration information, the computer processor determines whether to contact a predetermined contact based on the risk level and the personal medical information;
a storage that stores the personal medical information and the risk level; and
a portable device attached to the user, comprising:
an acceleration sensor that outputs user's abnormal movement signals;
a position receiver that calculates position information of the user;
a clock that outputs time information;
a portable transceiver that transmits the position information, the acceleration information based on the user's abnormal movement signals, and the time information to the computer; and
a portable processor that receives signals from the acceleration sensor, the position receiver, and the clock and that controls the portable transceiver,
wherein the computer, the storage, and the portable device are connected to each other via a computer network.

2. The system according to claim 1, wherein
the portable device further comprises a biological sensor that outputs biological information of the user,
the portable transceiver transmits the biological information, the position information, and the time information to the computer.

3. The system according to claim 2, wherein the computer processor further:
updates the personal medical information to append the biological information or the acceleration information upon receiving the biological information or the acceleration information of the user; and
recalculates the risk level based on the updated personal medical information.

4. The system according to claim 3, wherein the portable device further comprises an output device that outputs health information based on an instruction from the computer.

5. The system according to claim 4, wherein the computer processor further transmits the instruction when the received biological information or the acceleration information is within a notification range based on the risk level.

6. The system according to claim 4, wherein the computer processor:
determines a risk pattern that is applied where a predetermined combination of risk factors of the personal medical information is within notification ranges respectively, and
transmits the instruction based on the risk pattern when the personal medical information is updated.

7. The system according to claim 6, wherein the computer processor transmits the instruction based on the risk pattern periodically.

8. The system according to claim 4, wherein the output device is at least one of a display, a sound speaker, and a vibration device.

9. The system according to claim 4, wherein the computer processor:
causes the output device to output question information;
receives answer information in response to the question information; and
stores the answer information to the storage as a part of the personal medical information.

10. The system according to claim 4, wherein
the biological sensor comprises a pedometer, and
the computer processor causes the output device to output a warning of lack of exercise when a value indicated by the pedometer is within a notification range.

11. The system according to claim 4, wherein
the biological sensor comprises a blood-pressure gauge, and
the computer processor causes the output device to output a warning when a blood pressure while sleeping is not lower than the blood pressure in day time.

12. The system according to claim 4, wherein
the biological sensor comprises a blood-pressure gauge, and
the computer processor causes the output device to output a warning when a blood pressure indicated by the blood-pressure gauge is within a notification blood-pressure range.

13. The system according to claim 4, wherein the computer processor causes the output device to output a warning upon determining a gait abnormality based on a signal from at least one of the acceleration sensor, the position receiver, and a pedometer.

14. The system according to claim 4, wherein
the biological sensor comprises a microphone, and
the computer processor causes the output device to output a warning upon determining a language abnormality based on signals from the microphone.

15. The system according to claim 4, wherein
the biological sensor comprises an electrocardiograph, and
the computer processor causes the output device to output a warning upon determining an irregular heartbeat based on signals from the electrocardiograph.

16. The system according to claim 1, wherein
the portable device further comprises a plurality of buttons, and
the portable transceiver transmits an emergency operation information when the plurality of buttons are held down.

17. The system according to claim 1, wherein
the portable transceiver transmits cancel operation information when the portable processor receives a cancel operation of the user,
the computer processor stops a process to contact one or more suitable hospitals and/or the predetermined contact when the computer transceiver receives the cancel operation information.

18. The system according to claim 1, wherein the personal medical information includes medical record information received from at least one of a medical office, a health check result received from a medical office, and a genetic test result received from a genetic testing company.

19. The system according to claim 1, wherein the computer transceiver transmits supplemental information together with the position information and the time to one or more suitable hospitals and/or the predetermined contact when the computer processor contacts the predetermined contact.

20. The system according to claim 19, wherein
the computer processor further determines which of cerebral infarction or cerebral hemorrhage is more likely based on the personal medical information, and
the supplemental information includes the determination of higher possibility.

21. The system according to claim 19, wherein
the storage stores at least one of information items of physical address of the user, commuting route, information about a resident house, and accessibility of the resident house,
the computer processor determines whether an emergency status occurs at the resident house of the user by comparing between the position information and the physical address of the user, and
the computer transceiver transmits, when the computer processor determines that the emergency status occurs at the resident house of the user, at least one of the information about the resident house and the accessibility of the resident house as the supplemental information.

22. The system according to claim 19, further comprising:
a hospital database storage that stores hospital data including at least one of information items of name of hospital, location information of the hospital, repeatedly updated hospital's availability for urgent treatment, possibility of advanced medical treatment, and name of doctor, wherein
the computer processor selects nearest hospitals from the position of the user among the hospital data when the computer processor determine that the personal medical information indicates that the user does not have any history of cerebral infarction or cerebral hemorrhage, or the user does not have any history of cerebral infarction in a large vessel even if the user had cerebral infarction in a past and
the computer transceiver transmits the hospital data of the nearest hospitals as the supplemental information.

23. The system according to claim 19, further comprising:
a hospital database storage that stores hospital data including at least one of information items of name of hospital, location information of the hospital, repeatedly updated hospital's availability for urgent treatment at that time, possibility of advanced medical treatment, and name of doctor, wherein
the computer processor selects hospitals that serve the advanced medical treatment from the nearest hospitals the computer processor selects from the position of the user among the hospital data when that the personal medical information indicates that the user has a history of cerebral infarction in the large vessel,
the computer transceiver transmits the advanced medical treatment hospital data of the nearest hospitals as the supplemental information.

24. The system according to claim 1, wherein the predetermined contact includes at least one of a fire station, a hospital, a family contact, and a work place contact.

25. The system according to claim 1, wherein
the portable device is composed of a plurality of devices connecting through wired or wireless communication, and
the plurality of devices comprises at least one of a wearable device, a smart phone, a display, and a biological sensor.

26. The system according to claim 1, wherein the computer comprises a plurality of computer devices connected to the computer network each other.

27. A computer for monitoring and responding to a cerebrovascular accident, comprising:
a computer processor that:
receives personal medical information; and
calculates a risk level of a cerebrovascular accident based on the personal medical information; and
a computer transceiver, wherein
when receiving acceleration information from a portable device, the computer processor determines whether to contact a predetermined contact based on the risk level and the acceleration information.

28. A portable device attached to a user, comprising:
an acceleration sensor that outputs acceleration signals;
a position receiver that calculates position information of the user;
a clock that outputs time information;
a portable processor that obtains acceleration information from the acceleration sensor;
a portable transceiver that transmits the position information, acceleration information based on the acceleration signals, and time to a computer; and
an output device that outputs health information based on an instruction from the computer.

29. A method for monitoring a cerebrovascular accident, comprising:
receiving personal medical information by a computer;
calculating a risk level of cerebrovascular accident based on the personal medical information by the computer;
obtaining acceleration information based on acceleration signals from an acceleration sensor by a portable device;
determining by the computer, based on the acceleration information and on the risk level, whether to contact predetermined contact; and
contacting the predetermined contact when determining to contact predetermined contact.
